(19) United States
(12) Patent Application Publication
(10) Pub. No.: US 2008/0281251 A1
(43) Pub. Date: Nov. 13, 2008

(54) Medical Devices

(75) Inventor: Travis Yribarren, San Mateo, CA (US)

Correspondence Address: WORKMAN NYDEGGER, 1000 EAGLE GATE TOWER,, 60 EAST SOUTH TEMPLE, SALT LAKE CITY, UT, 84111, US

(73) Assignee: ABBOTT LABORATORIES, Abbott Park, IL (US)

(21) Appl. No.: 11/747,631

(22) Filed: May 11, 2007

(51) Int. Cl. A61N 1/30; A61M 29/00

(52) U.S. Cl. 604/19; 604/100.01

(57) ABSTRACT

A medical device may be constructed using one or more components. At least some of the medical device's components may be treated with a softening agent and/or a texturizing agent. For example, the medical device may include an expandable member that may be sized and configured to be expanded within a blood vessel, and the expandable member may be treated with a softening agent and/or a texturizing agent. Also, for example, the medical device may include a catheter, and the catheter may be treated with a softening agent and/or a texturizing agent. One exemplary softening and/or texturizing agent is a sulfuric acid solution, but other softening and/or texturizing agents may be used.

### BACKGROUND

1. Field of the Invention

The present invention generally relates to medical devices.

2. Description of Related Art

Coronary artery disease occurs when the coronary arteries become narrowed or blocked by a buildup of substances called “plaque.” This plaque buildup may lead to poor blood flow to the heart, which may cause chest pain. If not treated, coronary artery disease may result in a heart attack and, in many cases, death. In fact, coronary artery disease is a leading cause of death in the United States.

Angioplasty is a conventional procedure for treating coronary artery disease. In a typical angioplasty procedure, a medical device is inserted through a blood vessel, such as an artery in the patient's arm, groin or wrist. After insertion, the medical device is guided to a desired position. In particular, the medical device, such as a balloon catheter, typically includes a long flexible tube and an expandable member, such as a balloon, and the medical device may be guided through various blood vessels to a position in which the balloon is located within or near a narrowed portion of an artery. When in the desired position, the balloon may then be inflated to help compress the plaque deposits and/or widen the artery, which may help provide improved blood flow when the balloon is deflated and the medical device is removed. Unfortunately, in guiding the balloon to and from this position, the balloon may damage the blood vessels through which it passes.

If desired, a small tubular device called a “stent” may be placed at or near the compressed plaque deposits and/or the widened artery. For example, in some instances, a balloon catheter may carry a stent to a desired position. When the stent is in the desired position, the balloon may then be inflated to help expand the stent. For example, at least a portion of the balloon may be positioned within the stent's passageway and the inflation may exert a force against the stent's inner wall to expand the stent. Expanding the stent may help compress the plaque deposits and/or widen the artery. Desirably, the stent may remain to help prop the artery open, which may help maintain this improved blood flow and thus increase the success rate of the angioplasty procedures.

### SUMMARY

A need therefore exists for medical devices that eliminate or diminish the above-described and/or other disadvantages and problems.

One aspect is a medical system that may include a first medical device and a second medical device. The second medical device may include a stent, and the first medical device may be used to guide the stent to a desired position within a patient, such as within a lumen of a coronary artery, other arteries, other blood vessels, other tubular bodily structures, and the like. The second medical device, however, may be used to guide the stent to other positions within any other bodily structure having other characteristics. The first medical device may also be used to help implant the stent at the desired location. For example, the first medical device may include an expandable member, such as a balloon, which may be expanded to help implant the stent. The expandable member may be treated with a softening agent and/or a texturizing agent, which may facilitate retention of the stent at the desired location.

Another aspect is a medical device and at least a portion of the medical device may be guided to a desired position within a patient. For example, the medical device may include an expandable member, which may be guided to a desired position within a patient, such as within a lumen of a coronary artery, other arteries, other blood vessels, other tubular bodily structures, and the like. The expandable member, however, may be positioned in any other bodily structure having other characteristics. When in the desired position, the expandable member may be expanded. Afterwards, the expandable member may be collapsed, and the medical device may be removed from the patient. The expandable member may be sized and configured for use in an angioplasty procedure and/or for other suitable medical procedures or purposes.

Yet another aspect is a medical device that may include one or more components treated with a softening agent. The softening agent desirably softens at least a portion of the components, which may advantageously help reduce damage to a patient's body when the medical device is moved to a desired position within a patient. For example, the medical device may include an expandable member treated with a softening agent. The softening agent desirably softens at least a portion of the expandable member, which may advantageously help reduce damage to a patient's body when the expandable member is moved to a desired position within a patient. For example, the expandable member may be sized and configured to be inserted into and guided through blood vessels, and the softening of the expandable member may help reduce damage to the blood vessels.

Still another aspect is a medical device that may include one or more components treated with a texturizing agent. The texturizing agent can be used to texturize at least a portion of these components outer surfaces to make them rougher and less smooth. For example, the medical device may include an expandable member treated with a texturizing agent, which texturizes at least a portion of the expandable member's outer surface to make it rougher and less smooth.

Another aspect is a method for constructing at least a portion of a medical device. The medical device may include an expandable member, and the method may include applying an acid solution to an outer surface of the expandable member. The expandable member may be sized and configured to be expanded within a blood vessel. The expandable member may include a tube sized and configured to form at least a portion of the outer surface of the expandable member. The expandable member may include a balloon sized and configured to form at least a portion of the outer surface of the expandable member. The expandable member may be constructed from nylon. The acid solution may include a sulfuric acid solution.

Yet another aspect is a method for constructing at least a portion of a medical device. The medical device may include an expandable member, and the method may include applying a softening agent to at least a portion of the expandable member. The expandable member may be sized and configured to be expanded within a blood vessel. The at least a portion of the expandable member may include at least a portion of an outer surface of the expandable member. The at least a portion of the expandable member may include a tube. The at least a portion of the expandable member may include a balloon. The expandable member may be constructed from nylon. The expandable member may be constructed from a polyether block amide. The softening agent may include an acid solution. The softening agent may include a sulfuric acid solution.

Still another aspect is a method for constructing at least a portion of a medical device. The medical device may include a catheter, and the method may include applying a softening agent to at least a portion of the catheter. The at least a portion of the catheter may be sized and configured to be inserted into a blood vessel. The medical device may also include an expandable member, and the expandable member may be sized and configured to be expanded within a blood vessel. The at least a portion of the catheter may include at least a portion of an outer surface of the catheter. The catheter may be constructed from nylon. The catheter may be constructed from a polyether block amide. The softening agent may include an acid solution. The softening agent may include a sulfuric acid solution.

Another aspect is a method for constructing at least a portion of a medical device. The medical device may include an expandable member, and the method may include applying a texturizing agent to at least a portion of the expandable member. The expandable member may be sized and configured to be expanded within a blood vessel. The at least a portion of the expandable member may include at least a portion of an outer surface of the expandable member. The at least a portion of the expandable member may include a tube. The at least a portion of the expandable member may include a balloon. The expandable member may be constructed from nylon. The expandable member may be constructed from a polyether block amide. The texturizing agent may include an acid solution. The texturizing agent may include a sulfuric acid solution.

Yet another aspect is a method for constructing at least a portion of a medical device. The medical device may include a catheter, and the method may include applying a texturizing agent to at least a portion of the catheter. The at least a portion of the catheter may be sized and configured to be inserted into a blood vessel. The medical device may also include an expandable member, and the expandable member may be sized and configured to be expanded within a blood vessel. The at least a portion of the catheter may include at least a portion of an outer surface of the catheter. The catheter may be constructed from nylon. The catheter may be constructed from a polyether block amide. The texturizing agent may include an acid solution. The texturizing agent may include a sulfuric acid solution.

For purposes of summarizing, some aspects, advantages and features of some embodiments of the invention have been described in this summary. Not necessarily all of (or any of) these summarized aspects, advantages or features will be embodied in any particular embodiment of the invention. Some of these summarized aspects, advantages and features and other aspects, advantages and features may become more fully apparent from the following detailed description and the appended claims.

### BRIEF DESCRIPTION OF THE DRAWINGS

The appended drawings contain figures of preferred embodiments to further illustrate and clarify the above and other aspects, advantages and features of the present invention. It will be appreciated that these drawings depict only preferred embodiments of the invention and are not intended to limit its scope. The invention will be described and explained with additional specificity and detail through the use of the accompanying drawings in which:

FIG. 1 is a diagram illustrating an exemplary medical system including an exemplary medical device;

FIG. 2 is a diagram of a portion of the medical device shown in FIG. 1 with another exemplary medical device;

FIG. 3 is an exploded view of a portion of the medical devices shown in FIG. 2;

FIG. 4 is a flowchart illustrating an exemplary method; and

FIG. 5 is a flowchart illustrating an exemplary method.

### DETAILED DESCRIPTION

The present invention is generally directed towards a medical device. The principles of the present invention, however, are not limited to medical devices. It will be understood that, in light of the present disclosure, the medical device disclosed herein can be successfully used in connection with other types of devices. A detailed description of the medical device now follows.

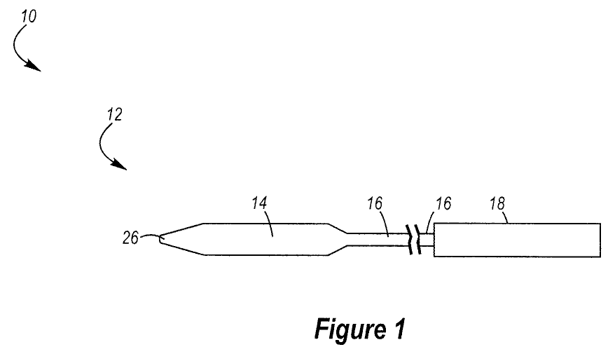

As shown in FIG. 1, a medical system 10 may include one or more medical devices, such as a medical device 12. The medical device 12 may include an expandable member 14, such as a balloon. The medical device 12 may also include a catheter 16, a handle 18, a guide wire (not shown) and/or other suitable components. The medical device 12, however, does not require any of these particular components. For example, the medical device 12 does not require the handle 18 and may include, for example, a port through which the medical device 12 and/or the catheter 16 may receive a liquid, a gas and/or other suitable substance to help expand the expandable member 14. One such exemplary port is an inflation hub. One exemplary inflation hub configuration may include a luer type fitting (or other suitable fitting) that may be used to couple an inflation device, such as a syringe or other suitable inflation device.

At least a portion of the medical device 12 may be guided to a desired position within a patient, such as within a lumen of a coronary artery, other arteries, other blood vessels, other tubular or non-tubular bodily structures, and the like. For example, at least a portion of the medical device 12 may be guided through various blood vessels to a position in which the expandable member 14 is located within or near a narrowed portion of an artery. It will be appreciated, however, that the expandable member 14 may be positioned in any other bodily structure having other characteristics.

When in the desired position, the expandable member 14 may be expanded. Afterwards, the expandable member 14 may be collapsed and the medical device 12 may be removed from the patient. For example, the expandable member 14 may include a balloon, such as a dilation balloon. The balloon may be inflated to help compress plaque deposits and/or widen an artery, which may help provide improved blood flow when the balloon is deflated and the medical device 12 is removed. Thus, in some embodiments, the medical device 12 may be used for angioplasty, such as percutaneous transluminal angioplasty and/or percutaneous transluminal coronary angioplasty. It will be appreciated, however, that the medical device 12 need not be used for angioplasty and may be used for other suitable medical procedures or purposes.

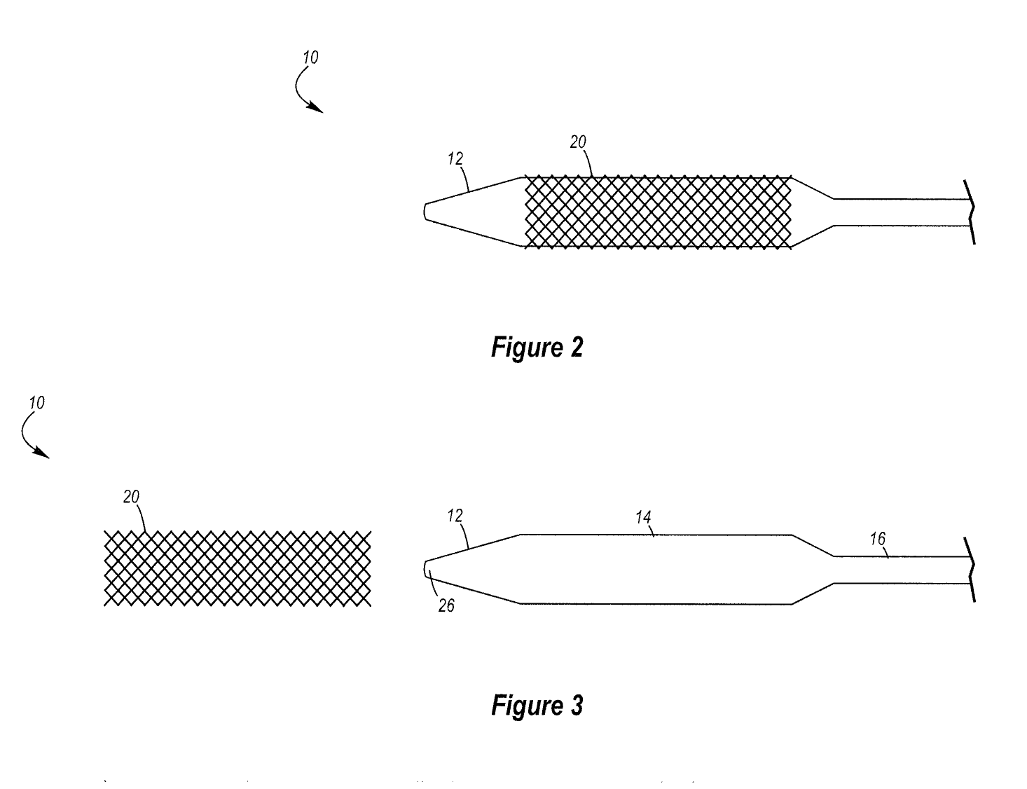

As shown in FIGS. 2-3, the medical system 10 may also include another medical device, such as a stent 20. The stent 20 can be constructed from metal mesh (such as steel mesh) formed into a tubular configuration having a cross section that is generally circular, square, rectangular, oval, oblong and/or other suitable shape. It will be appreciated, however, that the stent 20 may have a variety of other suitable constructions, configurations and/or shapes depending, for example, upon the particular stent. It will also be appreciated that the stent 20 need not be constructed from metal and may be constructed from other suitable materials.

At least a portion of the medical device 12 may be used to guide the stent 20 to a desired position within a patient, such as within a lumen of a coronary artery, other arteries, other blood vessels, other tubular or non-tubular bodily structures, other bodily structures and the like. For example, as shown in FIG. 2, the stent 20 may be connected to the medical device 12, and the stent and at least a portion of the medical device 12 may be guided through various blood vessels to a position in which the stent is located within or near a narrowed portion of an artery.

When in the desired position, the stent 20 may be implanted, and the medical device 12 may be removed from the patient. The stent 20 can be expanded to implant the stent at the desired position. For instance, in one embodiment, the medical device 12 may include an expandable member 14, such as a balloon, that may be expanded to help expand the stent 20. In particular, at least a portion of the balloon may be positioned within the stent's passageway and may be inflated to exert a force against the stent's inner wall to expand the stent. Expanding the stent 20 may help compress plaque deposits and/or widen an artery. Desirably, the stent 20 may remain to help prop the artery open, which may help provide improved blood flow when the balloon is deflated and the medical device 12 is removed. Thus, in some embodiments, the medical device 12 may be used for stent placement. It will be appreciated, however, that the stent 20 need not be balloon-expandable and may be self-expandable. It will also be appreciated that the stent 20 may be implanted using a variety of other suitable methods and/or at a variety of other suitable locations within a patient.

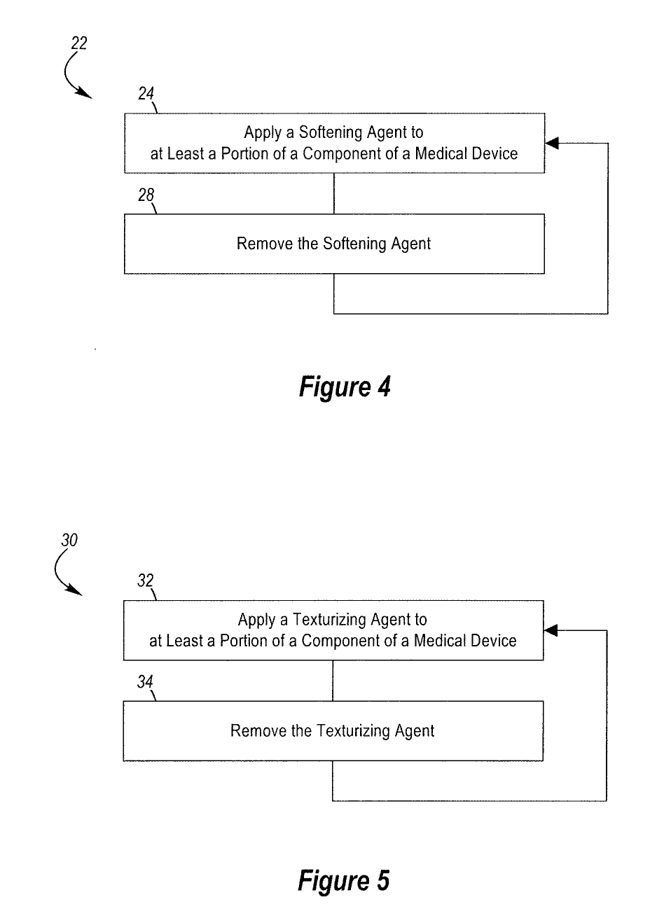

As shown in FIG. 4, a method 22 may be used to help construct a medical device. For example, the method 22 may be used to treat one or more components of an unassembled or partially assembled medical device 12, which may be later completely assembled to construct the medical device. Also, for example, the method 22 may be used to treat one or more components of a completely assembled medical device 12.

In further detail, at a block 24, a softening agent may be applied to at least a portion of one or more components of the medical device 12, such as the expandable member 14, the catheter 16, an end portion 26 and/or any other part of the medical device 12. For instance, in one embodiment, the softening agent may be applied to at least a portion of an outer surface of the expandable member 14, the catheter 16 and/or the end portion 26. If desired, the softening agent may be applied to an outer surface of a tube that is formed into the expandable member 14 and/or the catheter 16.

The application of the softening agent advantageously softens these components to make them less rigid and more flexible, which may advantageously help reduce damage to the patient's body when these components and/or the stent 20 are guided to a desired position within a patient. For example, when the softened components and/or the stent 20 are guided through the patient's blood vessels as mentioned above, the softened components' increased flexibility may help reduce the damage to those blood vessels. In addition, where the medical device 12 is used to help implant the stent 20, the softened components may facilitate better retention of the implanted stent 20. In particular, a softened expandable member 14 may facilitate better stent retention.

As shown in FIG. 4, at a block 28, the softening agent may be removed from the softened components of the medical device 12. For example, in one embodiment, the softened components may be constructed from nylon and the softening agent may comprise an acid solution, such as a sulfuric acid (H₂SO₄) solution, which may be flushed from the components at the block 28 using water and/or any other suitable liquid. As shown in FIG. 4, the blocks 24 and 28 may be repeated to further soften the components, if desired. It will be appreciated, however, that the softening agent need not comprise a sulfuric acid (H₂SO₄) solution and that other softening agents comprising other suitable acids and/or substances may be used. Other exemplary softening agents may include, but are not limited to, nitric acid; formic acid; acetic acids; mineral acids (such as boric acid, nitric acid, sulfuric acid, phosphoric acid, and hydrochloric acid); dicarboxylic acids (such as, malonic acid, succinic acid; glutaric acid, adipic acid, pimelic acid, suberic acid, azelaic acid, and sebacic acid); metallic salts (exemplary cations of the metallic salts may include aluminum, calcium, iron, magnesium, sodium, strontium, and zinc); diamines (such as, hexamethylene diamine); amides (such as, caprolactam and butyrolactam); adipoyl chloride; thermoplastic elastomers (such as, polyether block amides, and polyethylene terephthalates); and plasticizers. Exemplary plasticizers may include, but are not limited to, phthalate-based plasticizers (such as, bis(2-ethylhexyl) phthalate, diisononyl phthalate, bis(n-butyl)phthalate, butyl benzyl phthalate, diusodecyl phthalate, di-n-octyl phthalate, diisooctyl phthalate, diethyl phthalate, diisobutyl phthalate, di-n-hexyl phthalate, dibutyl phthalate, diethyl phthalate, and polyethylene terephthalates); adipate-based plasticizers (such as, bis(2-ethylhexyl)adipate, dimethyl adipate, monomethyl adipate, and dioctyl adipate); trimellitates (such as, trimethyl trimellitate, tri-(2-ethylhexyl) trimellitate, tri-(n-octyl,n-decyl) trimellitate, tri-(heptyl,nonyl) trimellitate, and n-octyl trimellitate); maleates (dibutyl maleate, and diisobutyl maleate); oxyalkylene glycols (such as, propylene glycol, ethylene glycol, diethylene glycol, dipropylene glycol, hexylene glycol, and tetraethylene glycol); sulfonamides (such as, N-ethyl toluene sulfonamide, N-(2-hydroxypropyl) benzene sulfonamide, and N-(n-butyl) benzene sulfonamide); fluoropolymers (such as, polytetrafluoroethylene); 2-pyrolodone; tributoxylethyl phosphate; polyurethanes; dibutyl sebacate; benzoates; polyethers; and alkyl citrates. It will also be appreciated that the softened components need not be constructed from nylon and may be constructed from other suitable materials having other suitable characteristics. Other exemplary materials that may be used to construct the softened components may include, but are not limited to, thermoplastic elastomers (such as, a polyether block amide, for instance, Pebax®), polyurethanes, polyethylene terephthalate (PET), polytetrafluoroethylene (PTFE), and extended polytetrafluoroethylene (ePTFE). In addition, bioabsorbable materials (such as, poly-L-lactide-co-glycolide, poly-dL-lactide-co-glycolide, polyester amide, chitosan, polybutylene terephthalate (PBT), 4-hydroxybutyrate, 3-hydroxybutyrate, and polyethylene glycol (PEG)) may be used to construct the softened components. It will be further appreciated that the softening agent need not be removed and thus the block 28 is optional depending, for example, upon the particular softening agent applied at the block 24.

As shown in FIG. 5, a method 30 may be used to help construct at least a portion of a medical device. For example, the method 30 may be used to treat one or more components of an unassembled or partially assembled medical device 12, which may be later completely assembled to construct the medical device. Also, for example, the method 30 may be used to treat one or more components of a completely assembled medical device 12.

In further detail, at a block 32, a texturizing agent may be applied to at least a portion of one or more components of the medical device 12, such as the expandable member 14, the catheter 16, the end portion 26 and/or any other part of the medical device 12. For instance, in one embodiment, the texturizing agent may be applied to at least a portion of an outer surface of the expandable member 14, the catheter 16 and/or the end portion 26. If desired, the texturizing agent may be applied to an outer surface of a tube that is formed into the expandable member 14 and/or the catheter 16. The application of the texturizing agent advantageously texturizes these components' outer surfaces to make them rougher and less smooth, which may facilitate better retention of an implanted stent 20 when, for example, the medical device 12 is used to help guide and/or implant the stent 20. In particular, a texturized expandable member 14 may facilitate better stent retention. Moreover, a texturized expandable member 14 may provide for better adhesion of substances (such as hydrophilic coatings and therapeutic agents) that may be applied to the texturized portions of the expandable member 14.

As shown in FIG. 5, at a block 34, the texturizing agent may be removed from the texturized components of the medical device 12. For example, in one embodiment, the texturized components may be constructed from nylon and the texturizing agent may comprise an acid solution, such as a sulfuric acid (H₂SO₄) solution, which may be flushed from the components at the block 34 using water and/or any other suitable liquid. As shown in FIG. 5, the blocks 32 and 34 may be repeated to further texturize the components, if desired. It will be appreciated, however, that the texturizing agent need not comprise a sulfuric acid (H₂SO₄) solution and that other texturizing agents comprising other suitable acids and/or substances may be used. Other exemplary texturizing agents may include, but are not limited to, nitric acid; formic acid; acetic acids; mineral acids (such as boric acid, nitric acid, sulfuric acid, phosphoric acid, and hydrochloric acid); dicarboxylic acids (such as, malonic acid, succinic acid; glutaric acid, adipic acid, pimelic acid, suberic acid, azelaic acid, and sebacic acid); metallic salts (exemplary cations of the metallic salts may include aluminum, calcium, iron, magnesium, sodium, strontium, and zinc); diamines (such as, hexamethylene diamine); amides (such as, caprolactam and butyrolactam); adipoyl chloride; thermoplastic elastomers (such as, polyether block amides, and polyethylene terephthalates); and plasticizers. Exemplary plasticizers may include, but are not limited to, phthalate-based plasticizers (such as, bis(2-ethylhexyl) phthalate, diisononyl phthalate, bis(n-butyl)phthalate, butyl benzyl phthalate, diisodecyl phthalate, di-n-octyl phthalate, diisooctyl phthalate, diethyl phthalate, diisobutyl phthalate, di-n-hexyl phthalate, dibutyl phthalate, diethyl phthalate, and polyethylene terephthalates); adipate-based plasticizers (such as, bis(2-ethylhexyl)adipate, dimethyl adipate, monomethyl adipate, and dioctyl adipate); trimellitates (such as, trimethyl trimellitate, tri-(2-ethylhexyl) trimellitate, tri-(n-octyl,n-decyl) trimellitate, tri-(heptyl,nonyl) trimellitate, and n-octyl trimellitate); maleates (dibutyl maleate, and diisobutyl maleate); oxyalkylene glycols (such as, propylene glycol, ethylene glycol, diethylene glycol, dipropylene glycol, hexylene glycol, and tetraethylene glycol); sulfonamides (such as, N-ethyl toluene sulfonamide, N-(2-hydroxypropyl) benzene sulfonamide, and N-(n-butyl) benzene sulfonamide); fluoropolymers (such as, polytetrafluoroethylene); 2-pyrolodone; tributoxylethyl phosphate; polyurethanes; dibutyl sebacate; benzoates; polyethers; and alkyl citrates. It will also be appreciated that the texturized components need not be constructed from nylon and may be constructed from other suitable materials having other suitable characteristics. Other exemplary materials that may be used to construct the texturized components may include, but are not limited to, thermoplastic elastomers (such as, a polyether block amide, for instance, Pebax®), polyurethanes, polyethylene terephthalate (PET), polytetrafluoroethylene (PTFE), and extended polytetrafluoroethylene (ePTFE). In addition, bioabsorbable materials (such as, poly-L-lactide-co-glycolide, poly-dL-lactide-co-glycolide, polyester amide, chitosan, polybutylene terephthalate (PBT), 4-hydroxybutyrate, 3-hydroxybutyrate, and polyethylene glycol (PEG)) may be used to construct the texturized components. It will be further appreciated that the texturizing agent need not be removed and thus the block 34 is optional depending, for example, upon the particular texturizing agent applied at the block 32.

The methods, systems and devices described above require no particular component, function or feature. Thus, any described component, function or feature—despite its advantages—is optional. Also, some or all of the described components, functions and features described above may be used in connection with any number of other suitable components, functions and features.

Although this invention has been described in terms of certain preferred embodiments, other embodiments apparent to those of ordinary skill in the art are also within the scope of this invention. Accordingly, the scope of the invention is intended to be defined only by the claims which follow.

What is claimed is:

1. A method for constructing at least a portion of a medical device, the medical device including an expandable member, the method comprising:
applying a softening agent to at least a portion of the expandable member, the expandable member being sized and configured to be expanded within a blood vessel.

2. The method as in Claim l, wherein the at least a portion of the expandable member comprises at least a portion of an outer surface of the expandable member.

3. The method as in claim 1, wherein the at least a portion of the expandable member comprises a tube.

4. The method as in claim 1, wherein the at least a portion of the expandable member comprises a balloon.

5. The method as in claim 1, wherein the expandable member is constructed from nylon.

6. The method as in claim 1, wherein the expandable member is constructed from a polyether block amide.

7. The method as in claim 1, wherein the softening agent comprises an acid solution.

8. The method as in claim 1, wherein the softening agent comprises a sulfuric acid solution.

9. A method for constructing at least a portion of a medical device, the medical device including a catheter, the method comprising:
applying a softening agent to at least a portion of the catheter, the at least a portion of the catheter being sized and configured to be inserted into a blood vessel.

10. The method as in claim 9, wherein the medical device further includes an expandable member, the expandable member being sized and configured to be expanded within a blood vessel.

11. The method as in claim 9, wherein the at least a portion of the catheter comprises at least a portion of an outer surface of the catheter.

12. The method as in claim 9, wherein the catheter is constructed from nylon.

13. The method as in claim 9, wherein the catheter is constructed from a polyether block amide.

14. The method as in claim 9, wherein the softening agent comprises an acid solution.

15. The method as in claim 9, wherein the softening agent comprises a sulfuric acid solution.

16. A method for constructing at least a portion of a medical device, the medical device including an expandable member, the method comprising:
applying a texturizing agent to at least a portion of the expandable member, the expandable member being sized and configured to be expanded within a blood vessel.

17. The method as in claim 16, wherein the at least a portion of the expandable member comprises at least a portion of an outer surface of the expandable member.

18. The method as in claim 16, wherein the at least a portion of the expandable member comprises a tube.

19. The method as in claim 16, wherein the at least a portion of the expandable member comprises a balloon.

20. The method as in claim 16, wherein the expandable member is constructed from nylon.

21. The method as in claim 16, wherein the expandable member is constructed from a polyether block amide.

22. The method as in claim 16, wherein the texturizing agent comprises an acid solution.

23. The method as in claim 16, wherein the texturizing agent comprises a sulfuric acid solution.

24. A method for constructing at least a portion of a medical device, the medical device including a catheter, the method comprising:
applying a texturizing agent to at least a portion of the catheter, the at least a portion of the catheter being sized and configured to be inserted into a blood vessel.

25. The method as in claim 24, wherein the medical device further includes an expandable member, the expandable member being sized and configured to be expanded within a blood vessel.

26. The method as in claim 24, wherein the at least a portion of the catheter comprises at least a portion of an outer surface of the catheter.

27. The method as in claim 24, wherein the catheter is constructed from nylon.

28. The method as in claim 24, wherein the catheter is constructed from a polyether block amide.

29. The method as in claim 24, wherein the texturizing agent comprises an acid solution.

30. The method as in claim 24, wherein the texturizing agent comprises a sulfuric acid solution.
